(19) United States
(12) Patent Application Publication
(10) Pub. No.: US 2009/0286989 A1
(43) Pub. Date: Nov. 19, 2009

(54) Process for High Purity Anastrozole

(76) Inventors: B. Vishnukant, Raichur (IN); Prashant Purohit, Deosugur (IN); K. Paparao, Deosugur (IN); Veereshappa Veereshappa, Deosugur (IN)

Correspondence Address: ABELMAN, FRAYNE & SCHWAB, 666 THIRD AVENUE, 10TH FLOOR, NEW YORK, NY, 10017, US

(21) Appl. No.: 12/224,965

(22) PCT Filed: Mar. 8, 2007
(86) PCT No.: PCT/IN2007/000090

§ 371 Date: Jan. 28, 2009

(30) Foreign Application Priority Data
Mar. 10, 2006 (IN) 430/CHE/2006

(51) Int. Cl. C07D 249/08

(52) U.S. Cl. 548/262.2

(57) ABSTRACT

The present invention relates to an improved process for the preparation of anastrozole having enhanced purity from crude anastrozole having isomeric impurity content up to less than 1%. The invention also relates to a process comprising steps of converting (3-cyanomethyl-5-methylphenyl) acetonitrile to 2-[(3-cyanodimethylmethyl)-5-methyl phenyl]- meiliyl propiononitrile (II) by C-alkylation, which is further converted into 2-[3-halomethyl-5-cyanodimethyl methyl)phenyl] methyl propiononitrile (ill) by radical bromination and further to crude anastrozole by reacting (III) with sodium salt of 1,2,4-triazole, purification of the crude anastrozole by preparing its acid addition salt, generating required final anaslrozole from the acid addition salt.

### FIELD OF INVENTION

The present invention relates to an improved process for the preparation of anastrozole having enhanced purity from crude anastrozole having isomeric impurity up to less than 1%.

### BACKGROUND AND PRIOR ART OF THE INVENTION

Anastrozole is an approved anticancer drug for the treatment of breast cancer. Several processes have been reported in the prior art for the preparation of Anastrozole. Patent documents EP 0 296 749 B1, FI 97804 C, HU 211142 B3, IE 65570 B1, NL 970012 I 2, PT 87720 B, ES 2063036 T T3, AU 605872 B2, CA 1337420 A1, IL 86499D D0, JP 2609290 B2, MX 9202876 A1, NO 170080 C, NZ 225037 A, ZA 8803691 A , U.S. Pat. No. 4,935,437, WO 2005/105 762 A, US2006/036950 and us2006/0276657 has been considered in entirety in this application.

### OBJECTS OF THE INVENTION

Main object of the invention is to provide an improved process for the preparation of anastrozole.

Another object of the invention is to provide a process for the preparation of crude anastrozole having isomeric impurity up to less than 1%.

Another object of the invention is to provide a process for the preparation of anastrozole having purity up to 99.85%, isomeric impurity of 0.03% with rest of all the impurities level being 0.11%

Yet another object of the invention is to provide a process devoid of column chromatography for the preparation of anastrozole.

It has been revealed in some of the prior art patented processes that the isomeric impurity accompanying anastrozole could not be reduced below 0.5% even by employing repeated crystallization. Also, the crude anastrozole prepared has isomeric impurity more than 1% to yield final pure anastrozole.

During process development for the preparation of anastrozole, Applicant observed that the increase in time period for the preparation of crude anastrozole increases the content of isomeric impurity in it. However, it has been revealed in WO 2005/105 762 A, published application that the isomeric impurity in the crude anastrozole could be decreased in the process by performing the coupling reaction with triazole alkali salt in DMF in combination with non-polar hydrocarbon solvent. Finally isomeric impurity of more than 1% in the crude anastrozole prepared could be only achieved by them.

It has been observed in our development, use of DMF as solvent in the absence of base for the preparation of crude anastrozole took longer time for the completion of coupling reaction ending up with more than 1% isomeric impurity.

Applicant's development for achieving crude anastrozole having low isomeric impurity is focused to decrease the reaction time in combination with the implementation of optimized work up condition for the reaction mixture.

Thus, Applicant achieved surprising result by using DMF as solvent in presence of a base followed by optimized working condition in the conversion of compound of formula (III) to crude anastrozole having isomeric impurity up to less than 1%, followed by its purification to obtain anastrozole of enhanced purity.

### SUMMARY OF THE INVENTION

The present invention relates to an improved process for the preparation anastrozole by obtaining 2-[(3-cyanodimethyl)-5-methyl phenyl]-methyl propinonitrile (II) from ( 3-cyanomethyl-5-methyl phenyl) acetonitrile (I), followed by bromination of (II) to obtain 2-[3-Bromomethyl-5-cynodimethyl methyl)ohenyl] methyl propionontrile (III), converting (III) to crude anastrozole having isomeric impurity up to less than 1% and finally purification of crude anastrozole to obtain anastrozole of enhanced purity.

### DETAILED DESCRIPTION OF THE INVENTION

In accordance with the object of the invention, there is provided an improved process for the preparation of anastrozole of formula (IV) having enhanced purity up to 99.85% with an isomeric impurity of 0.03%), comprising steps of:

a) bromination the compound of formula (II)

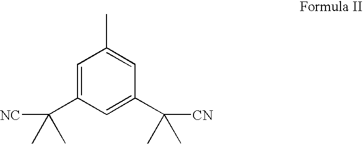

with a brominating agent, radical initiator in a non- polar organic solvent to obtain the bromo compound of formula (III)

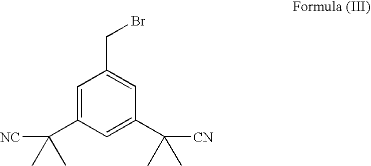

b) reacting the compound of formula (III), with alkali metal salt of triazole and alkali metal carbonate in a solvent DMF with or without non-polar solvent,

c) working up the reaction mixture of step (b) by pouring on to water and extracting with water immiscible organic solvent, distillation of organic solvent extract to obtain crude anastrozole having isomeric impurity up to less than 1%.

d) converting the crude anastrozole of step (c) to its acid addition salt by treating with organic acid or mineral acid in an aromatic hydrocarbon solvent containing optionally a polar solvent.

e) treating the acid addition salt of step (d) with a base to liberate the free base anastrozole and

f) extracting the free base anastrozole of step (e) with an organic solvent, concentrating and adding non polar hydrocarbon solvent to obtain anastrozole of enhanced purity.

An improved process of the present invention uses non-polar solvent for bromination selected from a group consisting of carbon tetrachloride and chlobenzene.

An improved process of the present invention uses brominating agent N-halosuccinimide, preferably N-bromosuccinimide.

An improved process of the present invention uses radical initiator in the step of bromination selected from a group consisting of benzolyperoxide and AIBN.

An improved process of the present invention uses optionally non-polar solvent in combination with DMF, preferably toluene or hexane solvent in the coupling reaction with alkali metal triazole.

An improved process of the present invention uses triazole salt selected from a group consisting of sodium triazole and potassium triazole.

An improved process of the present invention uses alkali carbonate selected from a group consisting of sodium carbonate and potassium carbonate.

An improved process of the present invention uses water immiscible solvent for extraction selected from a group consisting of toluene, methylene chloride disopropyl ether and chloroform, preferably toluene.

An improved process of the present invention uses organic acid selected from a group consisting of p-toluene sulphonic acid malefic acid, fumaric acid, oxalic acid and mineral acid selected from hydrochloric acid, phosphoric acid, sulphonic acid and hydrobromic acid for the preparation of acid addition salt.

An improved process of the present invention uses aromatic solvent toluene optionally with polar solvent selected from a group consisting of methanol, ethanol, isoproponal, acetonitrile and acetone for the preparation of acid addition salt.

The preparation of anastrozole is depicted in the following scheme represented below:

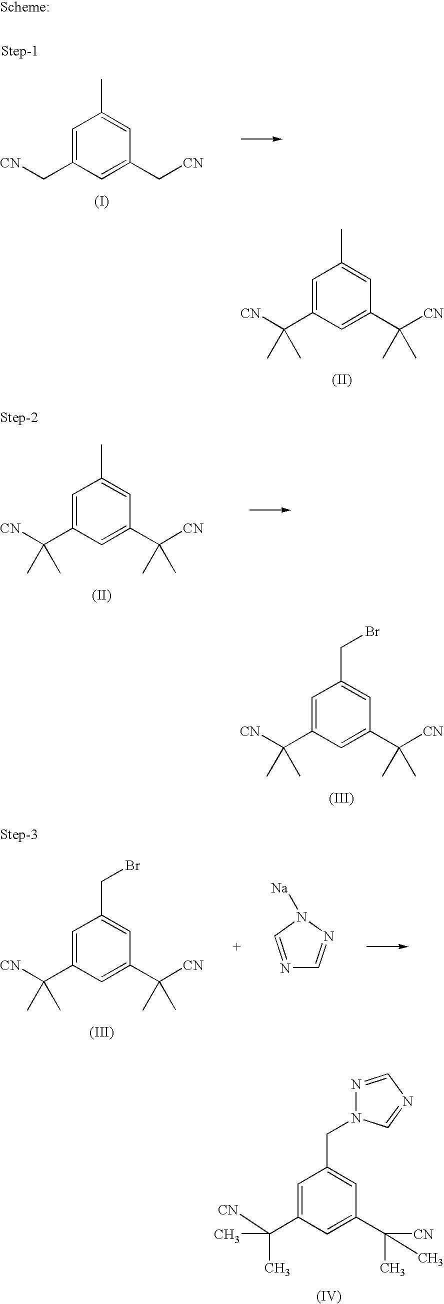

The following examples illustrate the invention and should not be construed to limit the scope of the present invention.

### EXAMPLE 1

### Preparation of 2-[(3-cyanodimethyl methyl)-5-methylphenyl]methyl propiononitrile (II)

(3-cyanomethyl-5-methyl phenyl) acetonitrile (I) is dissolved in N,N-dimethyl formamide at room temperature . . . Added potassium t-butoxide to it, stirred at about 15° C. for 4 to 5 hours, followed by the addition of methyl iodide, stirring for an hour to obtain the title compound (II).

### EXAMPLE 2

### Preparation of a,a,a′a′- tetra methyl 5-bromomethyl-1,3-Benzen acetonitrile of formula (III)

In a 3-necked flask, charge chlorobenzene (200 ml) a,a,a′a′- pentamethyl-1,3 Benzene diacetonitrile of formula (II), followed by N-bromo succinimide (98.5 g) and AIBN (2.0 g) the reaction mixture heated to reflux for an hour. Cool the reaction mixture to room temperature and maintain 10° to 15° C. for 2 hr. Filter the mass and dry the cake. To the dried cake, charge DM water (1090 ml) stir at room temperature for 2 h. Filter and dry the residue under vacuum at 45° to 50° C. to obtain the title compound.

### EXAMPLE 3

### Preparation of Crude Anastrozole

To the title compound of example 2 (50 g) in dry DMF (250 ml), anhydrous potassium carbonate (26 g) and triazole sodium salt (232 g) are added and the mixture stirred at room temperature for 45 min. Worked up the reaction mixture by adding water (1500 ml) and extraction with toluene (3×300 ml). Combined toluene layer is washed with water (1×300 ml), washed toluene layer is dried over anhydrous sodium sulphate, filtered and evaporated toluene to obtain crude anastrozole (40 g) having isomeric impurity 0.5%.

### EXAMPLE 4

### Preparation of Crude Anastrozole

To the title compound of example 2 (500 g) in dry DMF (250 ml), anhydrous potassium carbonate (260 g) and triazole sodium salt (2320 g) are added and the mixture stirred at room temperature for 45 min. Worked up the reaction mixture by adding water (15000 ml) and extraction with toluene (3×3000 ml). Combined toluene layer is washed with water (1×3000 ml), washed toluene layer is dried over anhydrous sodium sulphate, filtered and evaporated toluene to obtain crude anastrozole (450 g) having isomeric impurity 0.5%.

### EXAMPLE 5

### Preparation of Crude Anastrozole

To the title compound of example 2 (50 g) in dry DMF (250 ml) anhydrous potassium carbonate (26 g) and triazole sodium salt (232 g) are added and the mixture stirred at room temperature for 45 min. Worked up the reaction mixture by adding water (750 ml) and extracting with toluene (3×300 ml). Combined toluene layer is washed with water (1×300 ml). The washed toluene layer is dried over anhydrous sodium sulphate, filtered and evaporated toluene to obtain crude anastrozole (42 g) having isomeric impurity 4.52%.

### EXAMPLE 6

### Preparation of Crude Anastrozole

To the title compound of example 2 (50 g) in dry DMF (250 ml) anhydrous potassium carbonate (26 g) and triazole sodium salt (232 g) are added and the mixture stirred at room temperature for 45 min. Worked up the reaction mixture by adding water (950 ml) and extracting with toluene (3×300 ml). Combined toluene layer is washed with water (1×300 ml). The washed toluene layer is dried over anhydrous sodium sulphate, filtered and evaporated toluene to obtain crude anastrozole (42 g) having isomeric impurity 2.6%.

### EXAMPLE 7

### Preparation of Crude Anastrozole

To the title compound of example 2 (50 g) in dry DMF (250 ml) anhydrous potassium carbonate (26 g) and triazole sodium salt (232 g) are added and the mixture stirred at room temperature for 45 min. Worked up the reaction mixture by adding water (2500 ml) and extracting with toluene (3×300 ml). Combined toluene layer is washed with water (1×300 ml). The washed toluene layer is dried over anhydrous sodium sulphate, filtered and evaporated toluene to obtain crude anastrozole (42 g) having isomeric impurity 3.2%.

### EXAMPLE 8

### Preparation of Acid Addition Salt of Anastrozole from Crude Anastrozole

Crude Anastrozole (40 g) obtained in example 7 is taken in toluene (120 ml) and added p-toluene sulphonic acid monohydrate (25 g) with stirring. The mixture is heated to reflux for 30 mts and cooled to room temperature. The precipitated solid is filtered, washed with toluene, dried and crystallized to obtain anastrozole p-toluene sulphonic acid salt (30 g)

### EXAMPLE 9

### Preparation of pure Anastrozole from its p-toluene sulphonate salt

Anastrozole salt obtained in example 8 is suspended in DM H2O (200 ml) and stirred at room temperature for 15 minutes. To this ammonia solution is added dropwise to adjust the pH to about 9.00 to 10.00 Stir the mixture around 10° C. for 30 minutes. Extracted the reaction mixture with ethylacetate, concentrated ethylacetate soluble, added cyclohexane to obtain anastrozole having purity 99.85% and isomeric impurity of 0.03%.

### ADVANTAGES OF THE PRESENT INVENTION

1. The process of present invention circumvents chromatography in the purification of anastrozole of high purity.

2. The process of present invention provides crude anastrozole having minimum isomeric impurity, which has not been achieved in the earlier reported processes.

We claim:

1. An improved process for the preparation of anastrozole of formula (IV) having purity 99.85%, isomeric impurity of 0.03%, comprising steps of:
a) brominating the compound of formula (II) 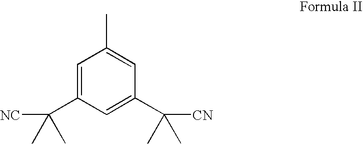
with a brominating agent, radical initiator in a non-polar organic solvent to obtain bromo compound of formula (III) 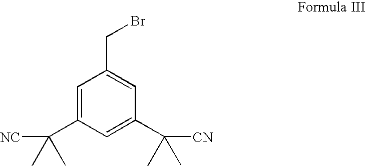
b) reacting the compound of formula (III) with alkali metal salt of triazole and alkali metal carbonate in a solvent DMF optionally containing non-polar solvent;
c) working up the reaction mixture of step (b) by pouring into water and extracting with water immiscible organic solvent, distilling organic solvent extract to obtain crude anastrozole having isomeric impurity up to less than 1%;
d) converting the crude anastrozole of step (c) to its acid addition salt by treating with organic acid or mineral acid in an aromatic hydrocarbon solvent optionally containing polar solvent,
e) treating the acid addition salt of step (d) with a base to liberate the free base anastrozole and
f) extracting the free base anastrozole of step (e) with ethylacetate, concentrating ethylacetate soluble, adding cyclohexane to obtain anastrozole of enhanced purity.

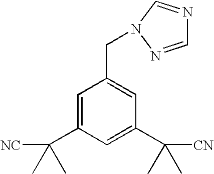

2. An improved process of claim 1, where in the non-polar solvent used in step (a) is selected from a group consisting of carbon tetrachloride and chlorobenzene.

3. An improved process of claim 1, where in the brominating agent used in step (a) is N-halosuccinimide selected from the group consisting of N-chlorosuccinimide and N-bromosuccinimide.

4. An improved process of claim 1, where in the radical initiator used in step (a) is selected from the group consisting of benzolyperoxide and AIBN.

5. An improved process of claim 1, wherein in step (b) the optionally used non-polar solvent is preferably toluene or hexane solvent.

6. An improved process of claim 1, where in the triazole salt used in step (b) is selected from a group consisting of sodium triazole and potassium triazole.

7. An improved process of claim 1, where in alkali carbonate used in step (b) is selected from a group consisting of sodium carbonate and potassium carbonate.

8. An improved process of claim 1, where in step (c) the water immiscible solvent used is selected from a group consisting of toluene, methylene chloride diisoopropyl ether and chloroform, preferably toluene.

9. An improved process of claim 1, where in step (d) the organic acid used is selected from a group consisting of p-toluene sulphonic acid maleic acid, fumaric acid, oxalic acid and mineral acid selected from hydrochloric acid, phosphoric acid, sulphonic acid and hydrobromic acid.

10. An improved process of claim 1, where in step (d) the aromatic solvent used is toluene and optionally used polar solvent is selected from group consisting of methanol, ethanol, isopropanol, acetonitrile and acetone.

11. An improved process of claim 1, where in step (e) the base used is aqueous ammoniacal solution.

12. An improved process for the preparation of anastrozole of formula (IV) having purity 99.85% substantially as herein described and shown in the examples and description.
